(19) United States
(12) Patent Application Publication
(10) Pub. No.: US 2006/0025666 A1
(43) Pub. Date: Feb. 2, 2006

(54) "Bulls-eye" surface electromyographic electrode assembly

(75) Inventors: Robert M. Getsla, San Jose, CA (US); Ronald P. Grevstad, San Jose, CA (US); Victor F. Simonyi, San Francisco, CA (US); Terah W. Smiley, San Francisco, CA (US)

Correspondence Address: BEYER WEAVER & THOMAS LLP, P.O. BOX 70250, OAKLAND, CA, 94612-0250, US

(73) Assignee: Kinesense, Inc.

(21) Appl. No.: 11/150,827

(22) Filed: Jun. 10, 2005

Related U.S. Application Data
(60) Provisional application No. 60/579,066, filed on Jun. 10, 2004.

(51) Int. Cl. A61B 5/04

(52) U.S. Cl. 600/372; 600/546

(57) ABSTRACT

A flexible, surface electromyographic electrode apparatus is provided for use on a surface of biological tissue to measure bio-electric signals thereof. The electrode apparatus includes a conductive signal electrode device having a signal contact adapted to directly contact the surface of the biological tissue, and a signal transmission portion electrically coupled to the signal contact. A conductive ground electrode device of the electrode apparatus includes a ground contact that is adapted directly contact the surface of the biological tissue. A ground transmission portion of the ground electrode device is electrically coupled to the ground contact. The ground contact is disposed substantially about the signal contact so as to substantially surround a peripheral edge of the signal contact when both are in contact with the tissue surface. An insulation washer device is further disposed between the signal contact and the ground contact to substantially prevent conductive contact therebetween.

### RELATED APPLICATION DATA

This claims priority under 35 U.S.C. § 119 to U.S. Provisional Application Ser. No. 60/579,066, which is incorporated herein by reference in its entirety for all purposes.

### TECHNICAL FIELD

The present invention relates, generally, to surface mounted electrode assemblies for measuring bioelectric signals, and specifically to surface mounted electromyographic electrodes assemblies.

### BACKGROUND ART

Surface electromyography electrode assemblies have a variety of industrial uses. Their primary application, however, is concentrated in the psychological, academic research and medical professional fields. For example, psychologists use EMG biofeedback to help patients learn to relax certain muscles, as an aid in overall relaxation. Academic researchers, on the other hand, use EMG measurements to study the impact of muscle contractions on human movement and biomechanics.

Medical professionals employ EMG biofeedback to help patients retrain damaged or atrophied muscles. This can include those recovering from neurological damage as well as those recovering from prolonged inactivity (e.g. post surgery).

Such retraining can be difficult, in part, because the human body will often engage and strengthen surrounding undamaged muscles as substitutes for damaged muscles in order to protect the damaged muscle from re-injury. This can be particularly problematic when the patient is not able to “sense” which muscle is contracting, the injured muscle or the one being substituted.

For example, the Vastus Medialis Oblique (VMO) and Vastus Lateralis (VL) muscles are both part of the quadriceps or “thigh” muscle group. Both muscles attach to the patella, or “kneecap”. Both muscles contract when a seated patient raises his/her leg from the perpendicular (to the ground) to the horizontal (fully extended) position. However, in addition to pulling the patella in the proximal (toward the hip joint) direction, these two muscles also pull in the medial (toward the midline of the body) and lateral (away from the midline of the body) directions. When the forces of these medial and lateral pulls are balanced, the patella “tracks” along its groove at the distal (away from the hip joint) end of the femur without excess wear on either side. Patients often have difficulty consciously choosing the relative amount of contraction between these two muscles.

When one of these two muscles is atrophied, for example the VMO, the body protects the atrophied muscle by over-utilizing a substitute, in this case the VL. As a result, the patella is pulled to one side, causing excessive wear. In addition, this substitution pattern tends to defeat the purpose of therapeutic exercises: instead of strengthening the targeted muscle (VMO) it can serve to increase the strength of the substituted VL muscle instead. The application of EMG biofeedback, however, has been shown to improve the patient's ability to perform their exercises while avoiding the muscle substitution effects.

Surface EMG

Surface EMG devices work by measuring, from the surface of the body, the electrical potential that develops across the surface of a muscle as it contracts. This potential is related to the force of the muscle contraction (i.e., as the muscle produces more force, either by increasing the contraction of its fibers or by contracting more of its fibers, the electrical potential increases, and vice versa). Since differential amplification is employed in all current commercial units, at least two electrodes and a reference ground electrode are required directly over the muscle.

High Impedance Signal Paths—Isolating In An Aqueous Medium

In order to rely on naturally occurring skin environments or aqueous solutions as the conductive medium, the electrode assembly of this design, which is the subject of our U.S. Pat. No. 6,865,409 to Gestla et al., herein incorporated by reference in its entirety for all purposes, uses the following design for electrode isolation. The design allows the subject's skin to “fill in the spaces” between the electrodes, providing a barrier to any signal “shorting” effects that might occur in the presence of moisture. The principle at work here is that conductivity through a salt solution (e.g. sweat, chlorinated pool water) is a function of the volume of the liquid between the electrodes. By pressing the electrode assembly against the skin, the volume of liquid surrounding the electrodes becomes vanishingly small. This approach relies on pressure rather than the viscosity of the conducting medium to ensure that no “bridging” between electrodes occurs.

Two or more high impedance signal paths will experience significant signal attenuation if both are exposed to the same aqueous solution. At present, most current designs require that the entire electrode assembly along with the measurement site be completely waterproofed. By contrast, in the design of the '394 application, the use of contact pressure isolation for the signal and ground contact areas reduces isolation requirements to individual waterproofing of the remaining sections of each signal path. Thus, contact pressure isolation yields a huge practical advantage in terms of daily use of SEMG for biofeedback. FIG. 4 (which is actually a side view of the present invention) shows the electrode apparatus 230 held in place over the tissue surface 210. In FIG. 4, the subject's tissues “fills in the spaces between adjacent contact portions 11 and 210, providing a barrier to any signal “shorting” effects that might occur in the presence of moisture. This effect can be achieved by pressing the electrode assembly against the surface of the skin. Note that the contact portions can be flush with the surface of the insulating material and still work by forcing the excess water out from the space between the conductive surfaces.

High Impedance Effects

A high impedance system using a “guard”, or voltage driven shield, can experience tribo-electric cable effects and antenna effects on the circuit board. These can be addressed by A) using low tribo-electric cabling and B) careful circuit board design.

Orientation of Signal Electrodes

Most current designs require that the signal electrodes be oriented in a line parallel to the fibers of the muscle being measured. The more accurate and selective the instrumentation, the more sensitive the measured signal is to this orientation. This can be quite inconvenient for the busy practitioner or patient, who must take additional time to properly align the electrodes. Also, the proper orientation can lead to an inconvenient orientation for the cabling which connects the electrode assembly to the control box.

It would be desirable, therefore, to provide an electrode assembly design that does not require alignment of the electrodes for optimal performance.

Redundant Signal Processing Circuitry

Present designs incorporate differential amplification, which involves calculating the difference between two input signals (Input Signal (1)−Input Signal (2)). External signals at a given amplitude tend to arrive at all signal electrodes simultaneously. These signals are then considered part of the “common mode” signal and are eliminated by differential amplification.

However, these input signals are already the result of a subtraction. They are the result of comparing the raw signal to the reference ground and taking the difference (signal (i)−ground). Substituting in the earlier formula, we have (signal (1)−ground)−(signal (2)−ground). The initial subtraction drops out and adds no value to the circuit.

It would be desirable, therefore, to design an emg first stage amplification circuit that takes full advantage of the comparison made by the amplifier between the raw signal and the ground reference.

### DISCLOSURE OF THE INVENTION

The present invention provides a flexible, surface electromyographic “bulls-eye” electrode apparatus for use on a surface of biological tissue to measure bio-electric signals thereof. The electrode apparatus includes a conductive signal electrode device having a signal contact adapted to directly contact the surface of the biological tissue to receive and transmit bio-electric signals. The signal electrode device further includes a signal transmission portion electrically coupled to the signal contact. A conductive ground electrode device includes a ground contact that is adapted to directly contact the surface of the biological tissue. A ground transmission portion of the ground electrode device is electrically coupled to the ground contact. The ground contact is disposed substantially about the signal contact so as to substantially surround a peripheral edge of the signal contact when both are in contact with the tissue surface. An insulation washer device is further disposed between the signal contact and the ground contact to substantially prevent conductive contact therebetween. The electrode apparatus further includes a substantially non-conductive, flexible, first sheet material disposed between the signal contact and the signal transmission portion, and between the ground contact and the ground transmission portion. This first sheet material substantially prevents conductive contact of the signal transmission portion and the ground transmission portion with the tissue surface.

Accordingly, signals from a source within the body migrate across the surface of the body in an expanding ring pattern. Signals whose source is external to the bulls-eye electrode assembly will always flow across the bulls-eye in the same configuration, regardless of point of origin. These external signal “rings” will decline in amplitude uniformly across the bulls-eye, so that the signal amplitude measured by the signal contact of the signal electrode device will equal, on average, the signal amplitude measured by the ground contact of the ground electrode device.

Target muscle signals, hence, emanating from underneath the bulls-eye, will radiate outward, in ring patterns that intersect the reference ground contact in consistent patterns. The reference ground electrode device will then detect a signal that is an average across a fixed, consistent range of signal rings. The relationship between the signal electrode target signal amplitude and the reference ground electrode target signal amplitude will be fixed and consistent, just as for multi point differential amplification arrangements.

In one specific embodiment, a conductive upper guard element is positioned substantially adjacent to and substantially over the signal electrode device. In this arrangement, the measured bio-electric signal passing therethrough is substantially shielded from ambient electric fields generated from sources above and external to the electrode apparatus. Similarly, a conductive lower guard element is positioned substantially adjacent to and substantially below at least a portion of the signal transmission portion such that the measured bio-electric signal passing therethrough is substantially shielded from ambient electric fields generated from sources below and external to the electrode apparatus.

In another configuration, a substantially non-conductive, flexible, second sheet material is positioned between the signal transmission portion and the upper guard element to substantially prevent conductive contact therebetween. Further, a substantially non-conductive, flexible, third sheet material is positioned between the signal transmission portion and the lower guard element to substantially prevent conductive contact therebetween.

In still another specific embodiment, the signal transmission portion of the signal electrode device includes a signal electrode footprint, and the upper guard element includes an upper guard footprint. The upper guard element is positioned and oriented such that when the electrode apparatus is operably mounted on the biological tissue, the upper guard footprint of the upper guard element at least extends over the signal electrode footprint. In other arrangements, the guard conductor footprint extends beyond at least a portion of the signal electrode footprint.

Yet another specific embodiment provides a substantially non-conductive, flexible, fourth sheet material positioned over the upper guard element that is mounted to the second sheet material in a manner enclosing the upper guard element therebetween. The first sheet material is mounted to the third sheet material in a manner enclosing the lower guard element therebetween.

In still another specific configurations, a second conductive lead extends through the first sheet material to electrically couple the signal contact portion to the signal transmission portion. Further, a second conductive lead extends through the first sheet material to electrically couple the ground contact portion to the ground signal transmission portion.

The signal transmission portion may include a contact head conductively coupled to the signal contact, and a signal transmission leg conductively coupled to the contact head. The ground transmission portion, in one arrangement, is U-shaped having a bight portion conductively coupled to the ground contact. The bright portion is configured to generally extend around the contact head of the signal transmission portion. A pair of ground transmission legs is provided with each conductively coupled to the bight portion. The ground transmission legs further are generally disposed on opposed sides of signal transmission portion. Each ground transmission leg is configured to be ground a spaced-apart locations.

In one specific embodiment, the signal transmission portion and the ground transmission portion are disposed within the same layer of the electrode apparatus. However, in another arrangement, the signal transmission portion and the ground transmission portion are separated by a substantially non-conductive, flexible, fifth sheet material positioned therebetween

In another aspect of the present invention, an electromyographic surface electrode assembly is provided for use on a surface of biological tissue. The electrode assembly includes a flexible, surface electromyographic electrode apparatus that includes a conductive signal electrode device having a signal contact adapted to directly contact the surface of the biological tissue to receive and transmit bio-electric signals. The signal electrode device further includes a signal transmission portion electrically coupled to the signal contact. A conductive ground electrode device is included having a ground contact to adapted directly contact the surface of the biological tissue. A ground transmission portion is electrically coupled to the ground contact, wherein the ground contact disposed substantially about the signal contact so as to substantially surround a peripheral edge of the signal contact when both are in contact with the tissue surface. An insulation washer device is disposed between the signal contact and the ground contact to substantially prevent conductive contact therebetween. The electrode apparatus further includes a substantially non-conductive, flexible, first sheet material disposed between the signal contact and the signal transmission portion, and between the ground contact and the ground transmission portion to substantially prevent conductive contact of the signal transmission portion and the ground transmission portion with the tissue surface. The electrode apparatus still further includes a conductive upper guard element positioned substantially adjacent to and substantially over the signal electrode device such that the measured bio-electric signal passing therethrough is substantially shielded from ambient electric fields generated from sources above and external to the electrode apparatus. A co-axial cable is provided having an inner conductor and an outer conductor shielding the inner conductor. At one portion of the co-axial cable, the inner conductor is electrically coupled to an opposite end of the signal transmission portion of the electrode device for transmission of the bio-electric signals. The outer conductor is electrically coupled to the upper guard element to substantially shield the inner conductor from the ambient electric fields generated from sources external thereto. Finally, a high impedance amplifier device is included having a signal input and a signal output. The signal input is electrically coupled to the inner conductor of the co-axial cable at another portion thereof for receipt of the transmitted bio-electric signals. The signal output is electrically coupled to the outer conductor of the co-axial cable, in a feedback loop, for receipt of at least a portion of the transmitted bio-electric signals, such that the voltage of the signals at the signal input of the high impedance amplifier device is maintained substantially equal to the voltage of the signals output from the signal output thereof.

### BRIEF DESCRIPTION OF THE DRAWINGS

The assembly of the present invention has other objects and features of advantage which will be more readily apparent from the following description of the best mode of carrying out the invention and the appended claims, when taken in conjunction with the accompanying drawing, in which:

FIGS. 1A-1C is an exploded perspective view of a “bulls-eye” flexible surface electromyographic electrode apparatus of an electrode assembly constructed in accordance with the present invention, and in particular illustrating the Signal Electrode Device.

FIGS. 2A-2C is also an exploded perspective view of a flexible surface electromyographic electrode apparatus of an electrode assembly constructed in accordance with the present invention, and in particular illustrating the Ground Electrode Device.

FIGS. 3A-3C is also an exploded perspective view of a flexible surface electromyographic electrode apparatus of an electrode assembly constructed in accordance with the present invention, and in particular illustrating the Guard Device.

FIG. 4 a cross-sectional view of the electrode apparatus of FIG. 1 operably mounted to biological tissue.

FIG. 5 is an enlarged, top perspective view, of the electrode apparatus of FIG. 1 coupled to a signal amplifier.

FIG. 6A is an exploded top perspective view of an alternative embodiment thereof.

FIG. 6B is an exploded bottom perspective view of the alternative embodiment of FIG. 6A.

FIG. 7A is a top plan view of the individual layers of the alternative embodiment of FIG. 6A.

FIG. 7B is a bottom plan view of the individual layers of the alternative embodiment of FIG. 6A.

FIG. 8 is a side elevation view, in cross-section, of the alternative embodiment of FIG. 6A.

### LEGEND

| Element | Number |
|---|---|
| Amplifier | 4 |
| Signal Input | 19 |
| Signal Output | 50 |
| Transmission Line | 5 |
| Signal Electrode Device (SED) | 10 |
| SED Contact Portion | 11 |
| SED Lead Portions | 12, 13 |
| SED Transmission Conductors | 14, 18 |
| Ground Electrode Device (GED) | 20 |
| GED Contact Portion | 21 |
| GED Lead Portion | 22 |
| GED Transmission Conductors | 23, 28 |
| Guard Device (GD) | 30 |
| GD Upper, Lower and Transmission | 31, 33, 38, 39 |
| Line Conductors | |
| Insulating Layers | 41, 42, 42, 44, 45 |
| Insulating Washers | 46, 47 |
| Electrode Assembly | 200 |
| Tissue Surface | 210 |
| Tissue | 220 |
| Electrode Apparatus | 230 |

### BEST MODE OF CARRYING OUT THE INVENTION

While the present invention will be described with reference to a few specific embodiments, the description is illustrative of the invention and is not to be construed as limiting the invention. Various modifications to the present invention can be made to the preferred embodiments by those skilled in the art without departing from the true spirit and scope of the invention as defined by the appended claims. It will be noted here that for a better understanding, like components are designated by like reference numerals throughout the various figures.

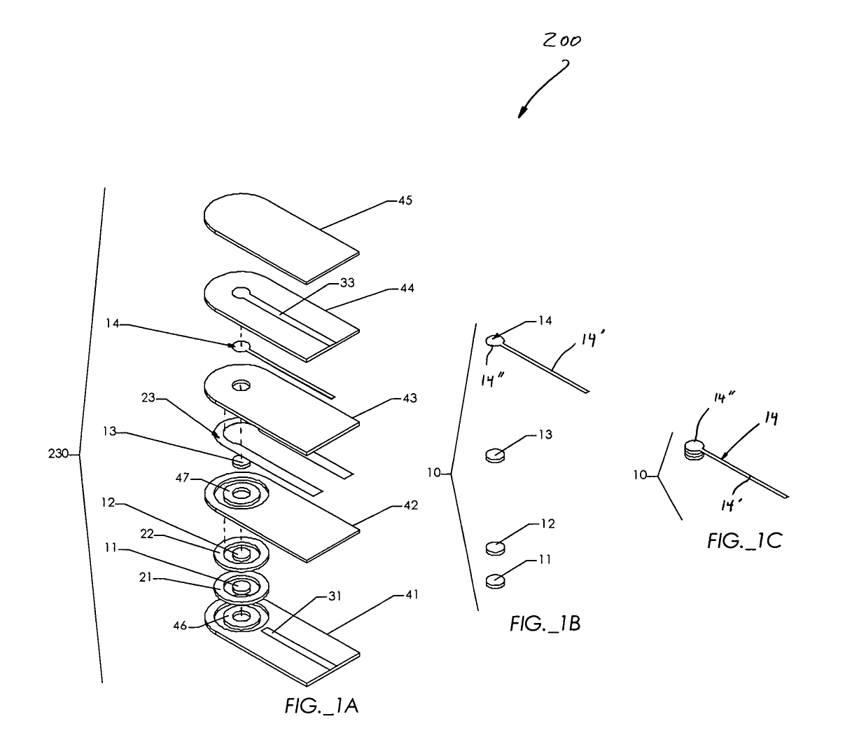

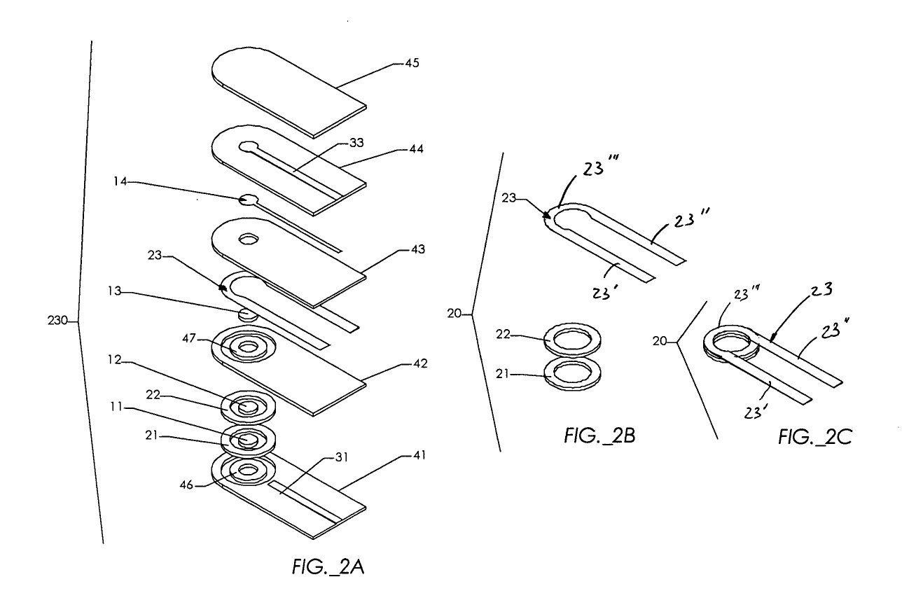

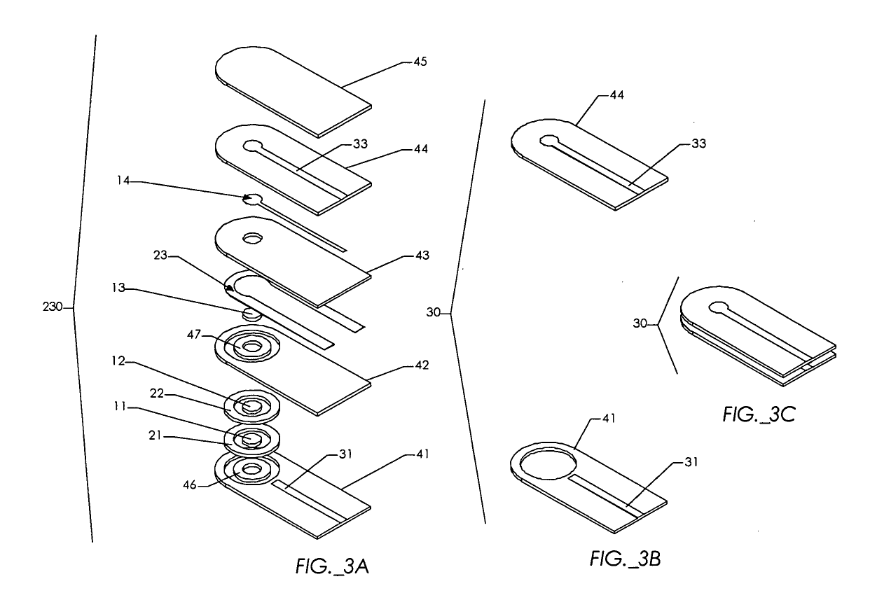

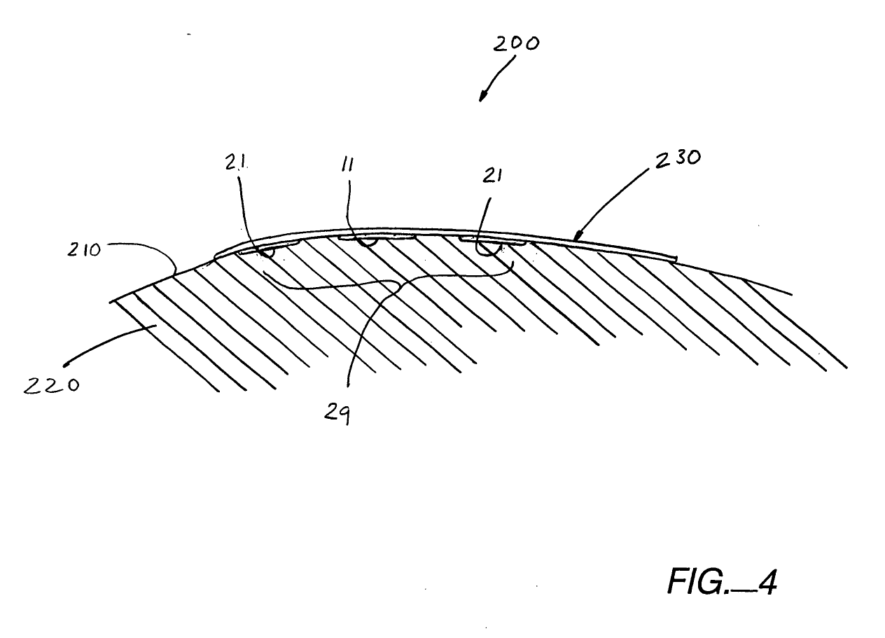

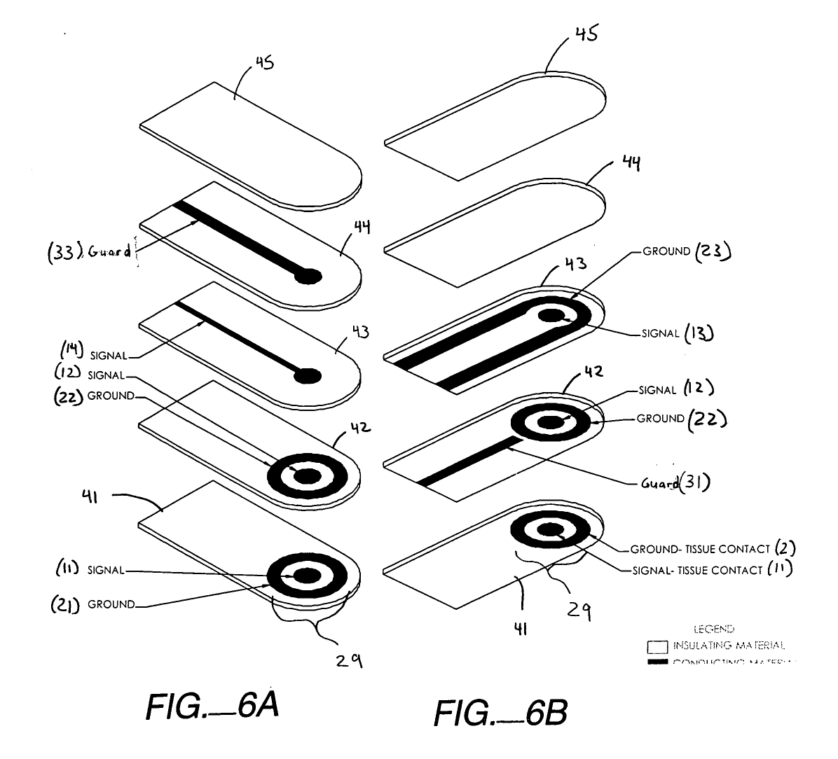

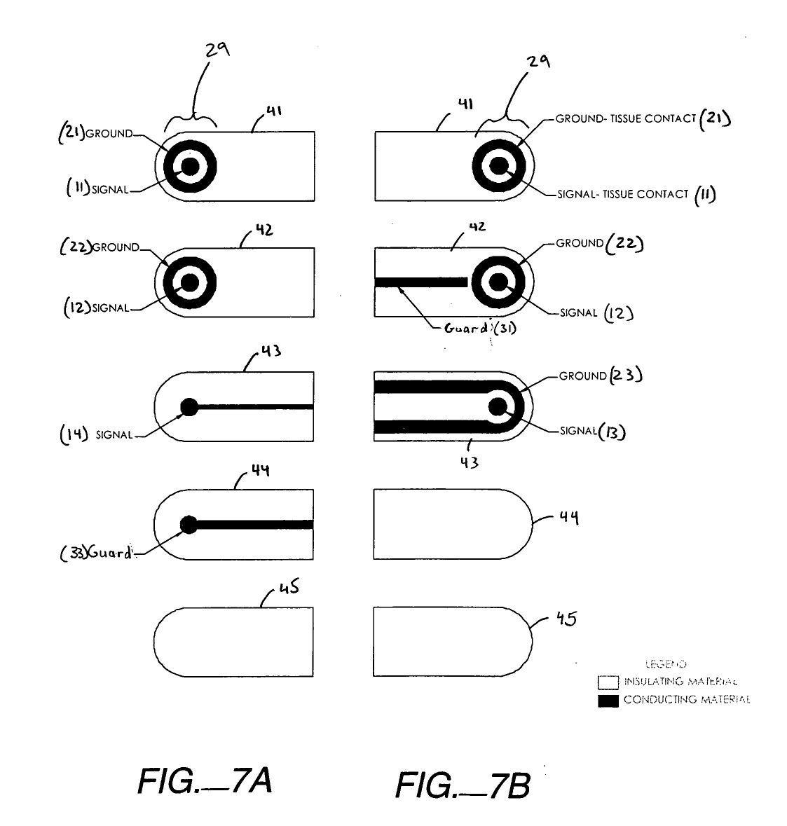

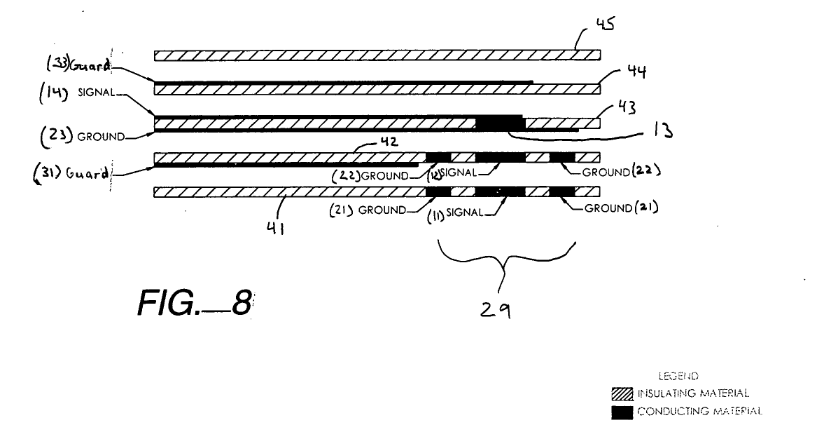

Referring now to FIGS. 1-5 and 8, a flexible, surface electromyographic “bulls-eye” electrode apparatus, generally designated 230, is disclosed for use on a surface 210 of biological tissue 220 (FIG. 220) to measure bio-electric signals thereof. The electrode apparatus 230 includes a conductive signal electrode device 10 (FIGS. 1B and 1C) having a signal contact 11 adapted to directly contact the surface 210 of the biological tissue 220 to receive and transmit bio-electric signals (FIG. 4). The signal electrode device 10 further includes a signal transmission portion 14 electrically coupled to the signal contact 11. The “bulls-eye” electrode apparatus 23 further includes conductive ground electrode device 20 (FIGS. 2B and 2C) that includes a ground contact 21 that is also adapted to directly contact the surface 210 of the biological tissue 220. A ground transmission portion 23 of the ground electrode device 20 is electrically coupled to the ground contact 21. The ground contact 21 is disposed substantially about the signal contact 11 so as to substantially surround a peripheral edge of the signal contact when both are in contact with the tissue surface 210 (forming a “bulls-eye” region 29 (FIGS. 4, 6 and 7)). An insulation washer device 46 is further disposed between the signal contact 11 and the ground contact 21 to substantially prevent conductive contact therebetween. The signal contact 11 and the ground contact 22 are adapted to directly contact the surface 210 of the biological tissue 220, in a concentric spaced-apart arrangement, to receive and transmit bio-electric signals measured sensed from the biological tissue 220, wherein each respective signal has an original respective first voltage and an original respective minute first current. Briefly, in accordance with the present invention, a guard device 30 (FIGS. 3B and 3C) is included that is disposed substantially adjacent to the signal electrode device 14 to substantially shield the same from ambient electric fields generated from sources both above and below (i.e., external to) the electrode apparatus 230. The guard device 30 includes a conductive upper guard element 33 positioned substantially adjacent to and substantially over the signal electrode device 14 such that the measured bio-electric signal passing therethrough is from substantially shielded from ambient electric fields generated from sources generally above and external to the electrode apparatus 230. Similarly, the guard device 30 includes a conductive lower guard element 31 positioned substantially adjacent to and substantially below at least a portion of the signal transmission portion 14 such that the measured bio-electric signal passing therethrough is substantially shielded from ambient electric fields generated from sources generally below and external to the electrode apparatus 230.

Further briefly, a plurality of substantially non-conductive, flexible, sheet materials (i.e., first sheet material 41, second sheet material 44, third sheet material 42 fourth sheet material 45, and fifth sheet material 43 ) are disposed between the respective circuits (i.e., guard elements 31 and 33, signal transmission portion 14 and ground transmission portion 23 ). Primarily, such sheet materials insulate the circuits from one another and from inadvertent contact the tissue surface 210.

The Kinesense “Bulls-eye” Design

In accordance with the present invention, hence, a surface electromyographic electrode apparatus is provided for use on a surface of biological tissue to measure bioelectric signals thereof. The conductive signal electrode device 10 is adapted to directly contact the surface 210 of biological tissue to receive and transmit bioelectric signals, via the disc shaped signal contact 11. The reference ground electrode device includes the ground contact 22, preferably in the shape of a thin washer that surrounds but does not touch the disc shaped signal contact 10. A first high impedance pre-amplifier 4 (FIG. 4) is included which receives input signals from a signal input connector 19 thereof, and references the signal from the reference ground electrode device 20. Accordingly, the present inventive design allows for the following, when placed over the target muscle.

Concentric Design

Signals from a source within the body move across the surface of the body in an expanding ring pattern. Signals whose source is external to the bulls-eye electrode apparatus 230 will always flow across the bulls-eye region 29 in the same configuration, regardless of point of origin. These external signal “rings” will decline in amplitude uniformly across the bulls-eye, so that the signal amplitude measured by the signal electrode device 10 will equal, on average, the signal amplitude measured by the ground electrode device 20.

The ground reference and signal voltages each can be decomposed into the sum of voltages from the target muscle and all other voltages. The bulls-eye reference ground and signal electrodes devices both detect the same voltage, on average, from non-target sources. The first stage amplifier 4 will see this non-target voltage as part of the zero potential baseline, to be excluded from the signal amplification.

In the bulls-eye design, the ground path (along the ground electrode device 20) doubles as a second signal path. The greater surface area of the washer lowers contact resistance sufficiently to eliminate the need for a high impedance path on this ground “signal” path. At the same time, the bulls-eye design eliminates the need for electrode orientation, since all external signals will now flow across the electrode apparatus 230 in the same configuration.

Target muscle signals, emanating from underneath the bulls-eye, will radiate outward, in ring patterns which intersect the ground reference ring in consistent patterns. The reference ground electrode device 20 will then see a signal that is an average across a fixed, consistent range of signal rings. The relationship between the signal electrode device target signal amplitude and the reference ground electrode device target signal amplitude will be fixed and consistent, just as for multi point differential amplification arrangements.

Referring back to FIGS. 3B and 3C, as mentioned, the guard device 30 includes the corresponding conductive guard device elements 31 and 33, each being positioned substantially adjacent and substantially below and above the signal electrode device 10, respectively, such that the respective measured bio-electric signal passing therethrough is substantially shielded from ambient electric fields generated from sources external to the electrode apparatus.

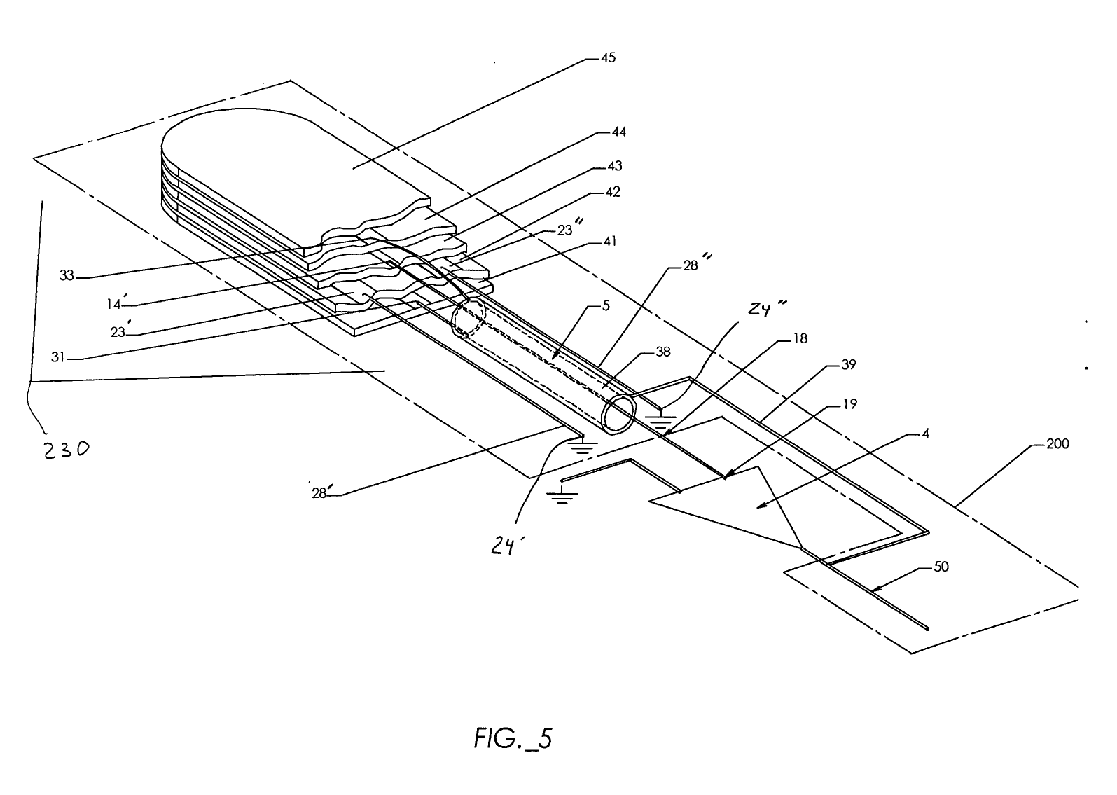

FIG. 5 best illustrates that the signal transmission conductor 18, at one portion thereof, is electrically coupled to a conductive leg 14′ of the corresponding signal device element 14 of the signal electrode device 10 for transmission of the bio-electric signal, while the guard conductor 38 is electrically coupled to the guard device elements 31 and 33. This arrangement functions to continuously shield the transmitted bio-electric signal from the ambient electric fields as it travels along the signal transmission conductor 5.

The signal transmission portion 14 includes a contact head 14″ and coupled to its conductive leg 14′ that define a signal electrode footprint. It will be appreciated that upper guard element 33 also includes an upper guard footprint that at least extends over the signal electrode footprint when the electrode apparatus 230 is positioned and operably mounted on the biological tissue surface 210. In one specific arrangement, the guard conductor footprint extends just beyond at least a portion of the signal electrode footprint to assure shielding. The footprint of the lower guard element 31 is also similarly sized and dimensioned.

The electromyographic surface electrode assembly 200 further includes a high impedance, first stage amplifier device, generally designated 4, having a signal input 19 and a signal output 50 (FIG. 5). The signal input 19 is electrically coupled to the signal transmission conductor 18 of the transmission line 5, at another portion thereof, for receipt of the transmitted bio-electric signals. The signal output 50 of the first stage amplifier device, on the other hand, is electrically coupled to the guard conductor 39, which is electrically coupled to guard conductor 38 of the transmission line 5, in a feedback loop, for receipt of at least a portion of the transmitted bio-electric signals. In this arrangement, the voltage of the signals at the signal input 19 of the high impedance, first stage amplifier device 4 is maintained substantially equal to the voltage of the signals output from the signal output thereof.

Accordingly, the electrode assembly of the present invention completes an outer “guard” circuit that protects the signal transmission circuit or conductor 10 from contamination by ambient electrical fields (for example, caused by fluorescent lighting, electrical wiring, etc.). This produces an interference resistant high impedance signal path with little or no antennae effect without the need for active amplification at the pickup site. As will be described in greater detail below, the physical absence of an active amplifier enables the construction of a uniformly, substantially flexible surface electrode apparatus that can easily conform to body contours. Further, since no active electronic components are present in or near the electrode apparatus itself, this electrode design is less expensive to manufacture than pre-amplified designs.

Another advantage of this EMG electrode assembly is that the application of a relatively high impedance amplifier will also result in a very low current along the signal path leading to the signal input to the high impedance, first stage amplifier device. The signal path leading to the signal input to the amplifier device itself can therefore be relatively high impedance (e.g., in the range of between about 10⁴ ohms to about 10⁶ ohms, compared to the impedance requirements of other designs) without introducing a significant voltage loss. This approach will therefore significantly increase the range of materials that can be used, including non-metals, to effectively and efficiently carry the signal from the source to the amplifier device.

Referring back to FIGS. 1A-1C, this electrode apparatus 230 of the electrode assembly 200 is preferably provided by a sandwich of four conductive circuitry layers (i.e., guard elements 31 and 33, signal transmission portion 14 and ground transmission portion 23 ) with insulative layers 41-45 (i.e., the flexiblefirst sheet material 41, second sheet material 44, third sheet material 42, fourth sheet material 45, and fifth sheet material 43) disposed correspondingly therebetween to prevent conductive contact. In addition, the signal transmission portion 14 of the signal electrode device 10 and the ground transmission portion 23 of the ground electrode device 20 are electrically connected to their corresponding signal contact 11 and ground contact 22 disk shaped contact elements 12 and 13 and conductive washer element 22, respectively. Such contact elements 12, 13 and conduct washer element 22 enable passage through insulative first sheet material 41, third sheet material 42 and fifth sheet material 43.

Insulative washer elements 46 and 47 insulate electrical contact between ground contact 21 and signal contact 11, and washer element 22 and contact element 12, respectively. Thus, the conductive circuit layers containing the signal electrode device 10 (FIG. 1), the ground electrode device 20 (FIG. 2), and the guard device 30 (FIG. 3) are electrically isolated from each other.

Briefly, while all washer elements and contact elements are shown having circular peripheries, other geometries are may be applied without departing from the true spirit and nature of the present invention. In fact, the peripheral edge geometries may even be mixed, and the contacts may be provided by point contacts or leads extending through the respective insulative layers.

To provide conductive contact with the surface of biological tissue 220, the conductive electrode signal and ground devices 10 and 20 each include a corresponding surface signal contact 11 and ground contact 21 at the exposed bottom of the second sheet material which are adapted to directly contact the target tissue 210. For the signal electrode device 10 of FIG. 1, corresponding conductive leads (signal contact 12 and disk element 13) extend through the insulative sheet materials 41, 42, 43 and 46, 47 to provide electrical coupling with a signal transmission portion 14 contained solely between the first insular layers 43-44. In a similar manner, for the ground electrode device 20 of FIG. 2, corresponding conductive lead (ground contact 21 and washer element 22) extends through the insulative sheet materials 41, 42 and 46, 47 to provide electrical coupling with a signal transmission portion 23 contained solely between the first insular layers 42-43. Hence, collectively, the signal electrode device 10 includes the signal contact 11, the conductive leads 12, 13, and the signal transmission portion 14 with its conductive leg 14′ (FIGS. 1B, 1C). As shown in FIG. 5, the conductive leg 14′ is then electrically coupled to the signal transmission conductor 18 of the transmission line 5.

The ground electrode device 20, on the other hand, includes the ground contact 21, the conductive lead 22, the U-shaped signal transmission portion 23. As best illustrated in FIGS. 2B and 2C, the U-shaped transmission portion 23 includes a bight portion 23′″ sized to extend around the corresponding signal conductive leads 12, 13 without contacting them. The bight portion 23′″ is coupled to a pair of opposed conductive legs 23′, 23″, which in turn, are electrically coupled to corresponding leads 28′, 28″. These leads can then be grounded at connections 24′, 24″ (FIG. 5) at spaced-apart locations.

Briefly, when two connections 24, 24″ are grounded, it can be electrically determined that all of the electrode connections are in place between the flexible electrode and the signal cable. This is performed by passing a very small DC current in from one “Ground” connection (e.g., 24′), through the “U”, and back out the other “Ground” connection (e.g., 24″), and thereby sense the DC continuity of the conductive material. If continuity between the two “Ground” connections 24′, 24″ is not sensed, an audio alarm could sound, such as a small beeper, to alert the user of the possibility of false EMG signal readings. In this instance, there could be other electrode connections that also are not complete through the connector between the flat electrode and the cable back to the amplifier 4, etc. Hence, by providing a pair of “Ground” terminals 24, 24″, the signal integrity can be monitored.

It will be appreciated that while the ground transmission portion 23 and the signal transmission portion 14 are shown and illustrated as being contained within separate layers (i.e., separated by fifth sheet material 43), this need not be the case. In fact, due to the “U-shape” of the ground transmission portion 23, the signal transmission portion 14 with its conductive leg 14′ can be positioned in-between and extending substantially parallel to the opposed conductive legs 23′, 23″, permitting these circuits can be disposed within the same layer.

It will further be appreciated that ground transmission portion 23 does not need to be U-shaped or have can be provided by a single conductive leg 23′ and ground connection 24′ (not shown). For example, the current U-shaped signal transmission portion 23 could be replace by a P-shaped or lollipop-shaped unit having a single conductive leg.

As best viewed in FIGS. 1 and 4, these thin surface contact portions 11 and 21 of the electrode devices 10 and 20 are spaced-apart along the bottom exposed surface of first sheet material 41. It will be appreciated that the contact portions, as well as their corresponding conductive leads 12-14, 18 and 19, and signal transmission portions 22, 23, 28, do not conductively contact any portion of the other electrode devices. Further, it will be understood that the non-conductive, sheet materials 41-47 are sufficiently insulative and disposed between the signal, ground and guard electrode devices 10, 20 and 30 to prevent such shorting.

Such sheet-like materials that provide non-conductive and flexible properties, as well as sufficient electrical isolation are abundant. However, it is also preferable that such materials be substantially impervious to moisture and bio-compatible, of course. Examples of these materials include, but are not limited to various kinds of plastic or silicone compounds.

Regarding the composition of the signal, ground and guard devices 10 Q 20 and 30, including the surface contact portions 11, 21 of the signal and ground devices, these materials of course must be conductive in nature. Common circuitry materials such as thin strips of metal or some other conductive material may be applied. However, since the circuit can still be a very high impedance circuit, it is not necessary for these circuitry layers conductor sections to be highly conductive materials. So, for example, the conductive sections could be made of conductive silicone, conductive plastics or other metal or non-metal materials of various conductivities that may enhance flexibility. Accordingly, such materials may be easily integrated, molded, adhered, etc. to the insulated sheet materials to essentially form a unitary fabrication. Another advantage of the invention is that it allows for an EMG electrode design that removes the need to use any metals as part of surfaces that will have direct contact with the user's skin. This will eliminate skin allergy problems associated with some metals such as nickel.

Further, the conductive material of the surface signal contact 11 and ground contact 21 and/or the corresponding conductive leads 12-13, 22 of the signal and ground devices 10 and 20 need not be the same material as either of the other conductive layers. For instance, the signal and ground contacts of the electrode devices may be composed of a more bio-compatible, conductive silicon material, while the corresponding signal transmission portions may be comprised of a more conductive metallic material. Also, the conductive leads 12-13, 22 need not be of the same material as the other conductive material.

The collective nine layers (i.e., circuitry layers 31, 23, 14, 33 and sheet material layers 41-45) plus the interior conductive leads 12, 13, 22 and washer elements 46 and 47 are bonded to each other to make a robust assembly that is impervious to moisture. Examples of suitable adhesives to adhere the sheet material to one another, while maintaining sufficient flexibility, include, but are not limited to, silicon rubber cements. Collectively, a thin, ribbon like flexible electrode structure is fabricated that can be operably mounted directly to the surface of moving muscular tissue. Accordingly, not only does the present invention provide a flexible EMG electrode apparatus 230 that can be shaped to fit or adhere to any body contour, but it also enables it to be imbedded in or attached to the inside of articles of clothing, without changes in appearance or comfort. It is even permissible to retain this device in the clothing during washing thereof.

Still another advantage of the invention is that it allows for a flexible electrode apparatus 230 that can be of any length, with the electrodes clustered at one end. In effect, the electrode assembly may replace some of the shielded cable transmitting the signal to the processing circuitry. Such a design will enhance the electrode assembly's ability to A) be incorporated in clothing and/or B) body contour.

In accordance with another aspect of this design, as shown in FIG. 4, the signal contact 11 and the ground contact 21 are mounted or attached to the bottom exposed surface of the second sheet material 41 in a manner that is flush with, slightly protruding from, or slightly recessed from the exposed bottom surface of the first sheet material 41. Thus, when the electrode apparatus 230 is held in place over the tissue surface 210 (FIG. 4), the subject's tissues “fills in the spaces” between the adjacent contact portions 11 and 21, providing a barrier to any signal “shorting” effects that might occur in the presence of moisture. The principle at work here is that conductivity through a salt solution (e.g. sweat, chlorinated pool water) is a function of the volume of the liquid between the electrodes; and that by pressing the electrode assembly against the skin, the volume of liquid surrounding the electrodes becomes vanishingly small. This approach, accordingly, relies on pressure rather than the viscosity of the conducting medium to ensure that no “bridging” between electrodes occurs. Such pressure may be applied, for instance, by elasticized fabric such as Spandex

This electrode design enables the fabrication of a flat, flexible electrode assembly structure that performs equally well whether the user is on land, in water, or perspiring heavily since, under most circumstances, no specialized conductive medium is required. This is not so of the current electrode designs that require a viscous conductive medium between the tissue and the electrode to avoid shorting between electrodes.

This electrode design relies on natural skin environments for the necessary conductivity at the skin surface. Accordingly, little or no skin preparation is required for proper functioning of the EMG electrode apparatus of the present invention. Only in circumstances where very dry skin creates very high skin impedance will any preparation be necessary, and then merely wetting the contact areas with any convenient aqueous solution—(e.g. tap water, saline, etc.) will be the only requirement. This approach will result in changes in the conductivity at the surface of the skin during and between applications. The impedance of the amplifier can be high enough, however, that the overall impedance of the circuit does not change materially. Therefore, the accuracy of the signal reading will not be materially affected.

A further advantage of the invention is that an EMG electrode can be built that is insensitive to heat, and can even be autoclaved for sterilization between uses.

As indicated above and as illustrated in FIG. 5, the signal transmission conductor 18 of the shielded signal transmission line 5 is electrically coupled to the signal transmission portion 14 of the corresponding signal electrode device 10 of the electrode apparatus 230, while the shield conductor 38 of the shielded signal transmission line 5 is electrically coupled to the corresponding guard device elements 31 and 33 thereof. Thus, a shield transmission signal circuit is constructed for the entire circuit path from the contact portion 11 of the corresponding electrode device 10 to the first stage amplifier 4 thereof to shield the signal electrode device 10 from unwanted signals from nearby ambient electrical fields (e.g. overhead lighting, etc.).

Briefly, FIG. 5 illustrates that the amplifier output is driving the guard device 30. It will be understood, however, that this will only apply if the amplifier 4 has a voltage gain of unity or one. The closer the amplifier voltage gain is to exactly one, the better. Since only a voltage gain of about 1 is achieved, the current is being amplified thousands of times. The amplifier 4, thus, is driving the guard device 30, and preventing the internal capacitance of the cable from “loading down” the EMG signal and in preventing contamination of the EMG signal from outside noise sources.

1. A flexible, surface electromyographic electrode apparatus for use on a surface of biological tissue to measure bio-electric signals thereof, said electrode apparatus comprising:
a conductive signal electrode device having a signal contact adapted to directly contact the surface of the biological tissue to receive and transmit bio-electric signals, and a signal transmission portion electrically coupled to the signal contact;
a conductive ground electrode device having a ground contact to adapted directly contact the surface of the biological tissue, and a ground transmission portion electrically coupled to the ground contact, said ground contact is disposed substantially about the signal contact so as to substantially surround a peripheral edge of the signal contact when both are in contact with the tissue surface; and
an insulation washer device disposed between the signal contact and the ground contact to substantially prevent conductive contact therebetween.

2. The electrode apparatus according to claim 1, further including:
a substantially non-conductive, flexible, first sheet material disposed between said signal contact and said signal transmission portion, and between said ground contact and said ground transmission portion to substantially prevent conductive contact of said signal transmission portion and said ground transmission portion with the tissue surface.

3. The electrode apparatus according to claim 2, further including:
a conductive upper guard element positioned substantially adjacent to and substantially over said signal electrode device such that the measured bio-electric signal passing therethrough is substantially shielded from ambient electric fields generated from sources above and external to said electrode apparatus.

4. The electrode apparatus according to claim 3, further including:
a substantially non-conductive, flexible, second sheet material positioned between said signal transmission portion and said upper guard element to substantially prevent conductive contact therebetween.

5. The electrode apparatus according to claim 4, wherein
said signal transmission portion of said signal electrode device includes a signal electrode footprint, and
said upper guard element includes an upper guard footprint, said upper guard element being positioned and oriented such that when the electrode apparatus is operably mounted on the biological tissue, the upper guard footprint of the upper guard element at least extends over the signal electrode footprint.

6. The electrode apparatus according to claim 5, wherein
said guard conductor footprint extends beyond at least a portion of the signal electrode footprint.

7. The electrode apparatus according to claim 3, further including:
a conductive lower guard element positioned substantially adjacent to and substantially below at least a portion of said signal transmission portion such that the measured bio-electric signal passing therethrough is substantially shielded from ambient electric fields generated from sources below and external to said electrode apparatus.

8. The electrode apparatus according to claim 7, further including:
a substantially non-conductive, flexible, second sheet material positioned between said signal transmission portion and said upper guard element to substantially prevent conductive contact therebetween; and
a substantially non-conductive, flexible, third sheet material positioned between said signal transmission portion and said lower guard element to substantially prevent conductive contact therebetween.

9. The electrode apparatus according to claim 8, further including:
a substantially non-conductive, flexible, fourth sheet material positioned over said upper guard element and mounted to said second sheet material in a manner enclosing said upper guard element therebetween, and
said first sheet material being mounted to said third sheet material in a manner enclosing said lower guard element therebetween.

10. The electrode apparatus according to claim 2, further including:
a second conductive lead extending through said first sheet material to electrically couple the signal contact portion to the signal transmission portion; and
a second conductive lead extending through said first sheet material to electrically couple the ground contact portion to the ground signal transmission portion.

11. The electrode apparatus according to claim 1, wherein
said signal transmission portion includes a contact head conductively coupled to said signal contact, and a signal transmission leg conductively coupled to said contact head; and
said ground transmission portion is U-shaped having a bight portion conductively coupled to said ground contact and generally extend around said contact head of the signal transmission portion, and a pair of ground transmission legs each conductively coupled to said bight portion, said ground transmission legs further being generally disposed on opposed sides of signal transmission portion.

12. The electrode apparatus according to claim 11, wherein
each ground transmission leg is configured to be ground a spaced-apart locations.

13. The electrode apparatus according to claim 9, wherein
said signal transmission portion includes a contact head conductively coupled to said signal contact, and a signal transmission leg conductively coupled to said contact head; and
said ground transmission portion is U-shaped having a bight portion conductively coupled to said ground contact and generally extend around said contact head of the signal transmission portion, and a pair of ground transmission legs each conductively coupled to said bight portion, said ground transmission legs further being generally disposed on opposed sides of signal transmission portion.

14. The electrode apparatus according to claim 13, wherein
said signal transmission portion and said ground transmission portion are disposed within the same layer of the electrode apparatus.

15. The electrode apparatus according to claim 13, further including:
a substantially non-conductive, flexible, fifth sheet material positioned between said signal transmission portion and said ground transmission portion.

16. An electromyographic surface electrode assembly for use on a surface of biological tissue to measure bio-electric signals thereof, said electrode assembly comprising:
a flexible, surface electromyographic electrode apparatus including
a conductive signal electrode device having a signal contact adapted to directly contact the surface of the biological tissue to receive and transmit bio-electric signals, and a signal transmission portion electrically coupled to the signal contact;
a conductive ground electrode device having a ground contact to adapted directly contact the surface of the biological tissue, and a ground transmission portion electrically coupled to the ground contact, said ground contact disposed substantially about the signal contact so as to substantially surround a peripheral edge of the signal contact when both are in contact with the tissue surface;
an insulation washer device disposed between the signal contact and the ground contact to substantially prevent conductive contact therebetween;
a substantially non-conductive, flexible, first sheet material disposed between said signal contact and said signal transmission portion, and between said ground contact and said ground transmission portion to substantially prevent conductive contact of said signal transmission portion and said ground transmission portion with the tissue surface; and
a conductive upper guard element positioned substantially adjacent to and substantially over said signal electrode device such that the measured bio-electric signal passing therethrough is substantially shielded from ambient electric fields generated from sources above and external to said electrode apparatus;
a co-axial cable having an inner conductor and an outer conductor shielding the inner conductor, at one portion of said co-axial cable, said inner conductor being electrically coupled to an opposite end of the signal transmission portion of the electrode device for transmission of said bio-electric signals, and said outer conductor being electrically coupled to the upper guard element to substantially shield the inner conductor from said ambient electric fields generated from sources external thereto; and
a high impedance amplifier device having a signal input and a signal output, said signal input being electrically coupled to the inner conductor of the co-axial cable at another portion thereof for receipt of the transmitted bio-electric signals, said signal output being electrically coupled to the outer conductor of the co-axial cable, in a feedback loop, for receipt of at least a portion of the transmitted bio-electric signals, such that the voltage of the signals at said signal input of the high impedance amplifier device is maintained substantially equal to the voltage of the signals output from said signal output thereof.

17. The electrode assembly according to claim 16, further including:
a substantially non-conductive, flexible, second sheet material positioned between said signal transmission portion and said upper guard element to substantially prevent conductive contact therebetween.

18. The electrode assembly according to claim 17, further including:
a conductive lower guard element positioned substantially adjacent to and substantially below at least a portion of said signal transmission portion, said lower guard element being electrically coupled to the outer conductor to substantially shield the inner conductor from said ambient electric fields generated from sources external thereto.

19. The electrode assembly according to claim 18, further including:
a substantially non-conductive, flexible, second sheet material positioned between said signal transmission portion and said upper guard element to substantially prevent conductive contact therebetween; and
a substantially non-conductive, flexible, third sheet material positioned between said signal transmission portion and said lower guard element to substantially prevent conductive contact therebetween.

20. The electrode assembly according to claim 19, further including:
a substantially non-conductive, flexible, fourth sheet material positioned over said upper guard element and mounted to said second sheet material in a manner enclosing said upper guard element therebetween, and
said first sheet material being mounted to said third sheet material in a manner enclosing said lower guard element therebetween.

21. The electrode assembly according to claim 20, wherein
said signal transmission portion includes a contact head conductively coupled to said signal contact, and a signal transmission leg conductively coupled to said contact head; and
said ground transmission portion is U-shaped having a bight portion conductively coupled to said ground contact and generally extend around said contact head of the signal transmission portion, and a pair of ground transmission legs each conductively coupled to said bight portion, said ground transmission legs further being generally disposed on opposed sides of signal transmission portion.

22. The electrode assembly according to claim 16, further including:
a second conductive lead extending through said first sheet material to electrically couple the signal contact portion to the signal transmission portion; and
a second conductive lead extending through said first sheet material to electrically couple the ground contact portion to the ground signal transmission portion.

23. The electrode assembly according to claim 16, wherein
said signal transmission portion includes a contact head conductively coupled to said signal contact, and a signal transmission leg conductively coupled to said contact head; and
said ground transmission portion is U-shaped having a bight portion conductively coupled to said ground contact and generally extend around said contact head of the signal transmission portion, and a pair of ground transmission legs each conductively coupled to said bight portion, said ground transmission legs further being generally disposed on opposed sides of signal transmission portion.

24. The electrode assembly according to claim 23, wherein
each ground transmission leg is configured to be ground a spaced-apart locations.
